SEQUENCE LISTING (28 sequences)

SEQ ID NO: 1 - DNA, 513 bp, Macaca fascicularis
agggctgtgcctgggggcagcagccctgcctgggctcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggattcgccagggtctgattttttacgagaagctactgggatcggatattttcacaggggagccttctctgctgcctgatagccctgtgggccagcttcatgcctccctactgggcctcagccaactcctgcagcctgagggtcaccactgggagactcagcagattccaagccccagtcccagccagccatggcagcgcctccttctccgcttcaaaatccttcgcagcctccaggcctttgtggctgtagctgcccgggtctttgcccatggagcagcaaccctgagtccctaa

SEQ ID NO: 2 - protein, 170 aa, Macaca fascicularis
RAVPGGSSPAWAQCQQLSQKLCTLAWSAHPLVGHMDLREEGDEETTNDVPHIQCGDGCDPQGLRDNSQFCLQRIRQGLIFYEKLLGSDIFTGEPSLLPDSPVGQLHASLLGLSQLLQPEGHHWETQQIPSPSPSQPWQRLLLRFKILRSLQAFVAVAARVFAHGAATLSP

SEQ ID NO: 3 - DNA, 854 bp, Macaca fascicularis
atatgggaactgaagaaagacgtttatgttgtagaattggactggtacccggatgcccctggagaaatggtggtcctcacctgtgacacccctgaagaagatggtatcacctggaccttggaccagagtggtgaggtcttaggctctggcaaaaccctgaccatccaagtcaaagagtttggagatgctggccagtacacctgtcacaaaggaggcgaggctctaagccattcactcctgctgcttcacaaaaaggaagatggaatttggtccactgatgttttaaaggaccagaaagaacccaaaaataagacctttctaagatgcgaggccaaaaattattctggacgtttcacctgctggtggctgacgacaatcagtactgatctgacattcagtgtcaaaagcagcagaggctcttctaacccccaaggggtgacgtgtggagccgttacactctctgcagagagggtcagaggggacaataaggagtatgagtactcagtggagtgccaggaggacagtgcctgcccagccgctgaggagaggctgcccattgaggtcatggtggatgccattcacaagctcaagtatgaaaactacaccagcagcttcttcatcagggacatcatcaaacccgacccacccaagaacttgcagctgaagccattaaagaattctcggcaggttgaggtcagctgggagtaccctgacacctggagtactccacattcctacttctccctgacattctgcatccaggtccagggcaagagcaagagagaaaagaaagatagaatcttcacagacaagacctcagccacggtcatctgccgcaaaaatgccagctttag

SEQ ID NO: 4 - protein, 306 aa, Macaca fascicularis
IWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQVEVSWEYPDTWSTPHSYFSLTFCIQVQGKSKREKKDRIFTDKTSATVICRKNASFSVQAQDRYYSSSWSEWASVPCS

SEQ ID NO: 5 - DNA, 602 bp, Macaca fascicularis
agaaacctctccgtggccaccccaggcccagaaatgttcccgtgccttcaccactcccaaaacctgctgaaggccgccagcaacacgcttcagaaggccagacaaattctagaattttacccttgcacttctgaagagattgatcatgaagatatcacaaaagataaaaccagcacagtagaggcctgtttaccattggaattaatcaagaatgagagttgcctaaattccagagagacttctttcataactaatgggagttgcctggcctccagaaagacctcttttatgatggccctgtgccttaggagtatttatgaagacttgaagatgtaccaagtggagttcaagaccatgaatgcaaagcttctgagggatcctaagaggcagatctttctagatcaaaacatactgggagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaaagggc

SEQ ID NO: 6 - protein, 197 aa, Macaca fascicularis
RNLSVATPGPEMFPCLHHSQNLLKAASNTLQKARQILEFYPCTSEEIDHEDITKDKTSTVEACLPLELIKNESCLNSRETSFITNGSCLASRKTSFMMALCLRSIYEDLKMYQVEFKTMNAKLLRDPKRQIFLDQNILGVIDELMQALNFNSETVPQKSSLEEPDFYKTKIKLCILLHAFRIRAVTIDRVMSYLNAS

SEQ ID NO: 7 - DNA, 30 bp, Artificial Sequence
  chemically synthesized elasti linker sequence
gttcctggagtaggggtacctggggtgggc

SEQ ID NO: 8 - protein, 10 aa, Artificial Sequence
  chemically synthesized elasti linker sequence
VPGVGVPGVG

SEQ ID NO: 9 - DNA, 35 bp, Artificial Sequence
  chemically synthesized ARC1626 aptamer
acaggcaaguaauuggggagugcgggcggggugut

SEQ ID NO: 10 - DNA, 35 bp, Artificial Sequence; identical to SEQ ID NO: 9
  chemically synthesized ARC1626 aptamer

SEQ ID NO: 11 - DNA, 32 bp, Artificial Sequence
  chemically synthesized 5′ primer
agatgtgtcaccagcagntggtcatctcttgg

SEQ ID NO: 12 - DNA, 30 bp, Artificial Sequence
  chemically synthesized 3′ primer
gttttgcttaatatcttctacttttcctcc

SEQ ID NO: 13 - DNA, 31 bp, Artificial Sequence
  chemically synthesized 5′ primer
ggttttccctggtttttctggcatctcccct

SEQ ID NO: 14 - DNA, 27 bp, Artificial Sequence
  chemically synthesized 3′ primer
tcctggatcagaacctaactgcagggc

SEQ ID NO: 15 - DNA, 26 bp, Artificial Sequence
  chemically synthesized 5′ primer
agatttgagaagaaggcaaaaagatg

SEQ ID NO: 16 - DNA, 31 bp, Artificial Sequence
  chemically synthesized 3′ primer
tctgagtgccatccttgagctaatggcttta

SEQ ID NO: 17 - DNA, 31 bp, Artificial Sequence
  chemically synthesized 5′ primer
gctgttgctgctgtcctggacagctcagggc

SEQ ID NO: 18 - DNA, 29 bp, Artificial Sequence
  chemically synthesized 3′ primer
agctgctgcctttagggactcagggttgc

SEQ ID NO: 19 - DNA, 24 bp, Artificial Sequence
  chemically synthesized 5′ primer
tcgggacaattataaaaatgtggc

SEQ ID NO: 20 - DNA, 22 bp, Artificial Sequence
  chemically synthesized 3′ primer
cctcgctttttaggaagcattc

SEQ ID NO: 21 - DNA, 25 bp, Artificial Sequence
  chemically synthesized 5′ primer
aaaatgtggccccctgggtcagcct

SEQ ID NO: 22 - DNA, 22 bp, Artificial Sequence
  chemically synthesized 3′ primer
ttttaggaagcattcagatagc

SEQ ID NO: 23 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 24 - protein, 319 aa, Macaca fascicularis
WFSLVFLASPLMAIWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQVEVSWEYPDTWSTPHSYFSLTFCIQVQGKSKREKKDRIFTDKTSATVICRKNASFSVQAQDRYYSSSWSEWASVPCS

SEQ ID NO: 25 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 26 - protein, 180 aa, Macaca fascicularis
LLLLSWTAQGRAVPGGSSPAWAQCQQLSQKLCTLAWSAHPLVGHMDLREEGDEETTNDVPHIQCGDGCDPQGLRDNSQFCLQRIRQGLIFYEKLLGSDIFTGEPSLLPDSPVGQLHASLLGLSQLLQPEGHHWETQQIPSPSPSQPWQRLLLRFKILRSLQAFVAVAARVFAHGAATLSP

SEQ ID NO: 27 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 253 aa, Macaca fascicularis
MWPPGSASQPPPSPATATGLHPAARPEPLQCRLSMCPARSLLLVATLVLLDYLSLARNLSVATPGPEMFPCLHHSQNLLKAASNTLQKARQILEFYPCTSEEIDHEDITKDKTSTVEACLPLELIKNESCLNSRETSFITNGSCLASRKTSFMMALCLRSIYEDLKMYQVEFKTMNAKLLRDPKRQIFLDQNILGVIDELMQALNFNSETVPQKSSLEEPDFYKTKIKLCILLHAFRIRAVTIDRVMSYLNAS

(19) United States
(12) Patent Application Publication
(10) Pub. No.: US 2009/0142855 A1
(43) Pub. Date: Jun. 4, 2009

(54) Polynucleotides and Polypeptides of the IL-12 Family of Cytokines

(76) Inventors: Wei Tang, Dorchester, MA (US); Yuxun Wang, Shrewsbury, MA (US); Shuhao Zhu, Lincoln, MA (US)

Correspondence Address: MINTZ, LEVIN, COHN, FERRIS, GLOVSKYAND POPEO, P.C., ONE FINANCIAL CENTER, BOSTON, MA, 02111, US

(21) Appl. No.: 11/988,079

(22) PCT Filed: Jun. 30, 2006
(86) PCT No.: PCT/US2006/025656

§ 371 Date: Dec. 10, 2008

Related U.S. Application Data
(63) Continuation-in-part of application No. 60/696,449, filed on Jun. 30, 2005.

(51) Int. Cl. G01N 33/566; C12N 15/11; C12N 15/00; C12N 5/08; C12P 21/00; C07K 14/54; C07K 16/18

(52) U.S. Cl. 436/501; 536/23.5; 435/320.1; 435/366; 435/69.52; 530/351; 530/387.9

(57) ABSTRACT

The present invention relates to the primate family of cytokines. Provided herein are polynucleotides encoding such polypeptides, and reagents useful for producing the encoded polypeptides in a host source, and for identifying compounds which bind and modulate the activity of the encoded polypeptides. Also provided are reagents useful for the diagnosis or treatment of inflammatory and/or autoimmune related diseases in primates.

### REFERENCE TO RELATED APPLICATIONS

This non-provisional patent application claims priority under 35 U.S.C. § 119(e) to the following provisional application: U.S. Provisional Patent Application Ser. No. 60/696,449 filed on Jun. 30, 2005 which is herein incorporated by reference in its entirety.

### FIELD OF INVENTION

The present invention relates generally to the field of polynucleotides and polypeptides, more particularly to nucleic acids encoding members of the IL-12 family of cytokines and purified polypeptides derived therefrom. This invention also provides reagents for producing purified proteins of the IL-12 family of cytokines, and antagonistic compounds against the same, for research, diagnostic and therapeutic uses.

### BACKGROUND OF THE INVENTION

The immune response in mammals is based on a series of complex cellular interactions called the “immune network.” In addition to the network-like cellular interactions of lymphocytes, macrophages, granulocytes, and other cells, soluble proteins known as lymphokines, cytokines, or monokines play a critical role in controlling these cellular interactions. Cytokine expression by cells of the immune system plays an important role in the regulation of the immune response. Most cytokines are pleiotropic and have multiple biological activities including antigen-presentation; activation, proliferation, and differentiation of CD4+ cell subsets; antibody response by B cells; and manifestations of hypersensitivity. Cytokines are implicated in a wide range of degenerative or abnormal conditions which directly or indirectly involve the immune system and/or hematopoietic cells. An important family of cytokines is the IL-12 family which includes, e.g., IL-12, IL-23, IL-27, and p40 monomers and p40 dimers. IL-23 is a covalently linked heterodimeric molecule composed of the p19 and p40 subunits, each encoded by separate genes. IL-12 is also a covalently linked heterodimeric molecule and consists of the p35 and p40 subunits, each encoded by separate genes. Thus, IL-23 and IL-12 both have the p40 subunit in common.

p40, p35, and p19 genes have been previously isolated and purified from multiple organisms including mouse, rat, dog, pig, and humans. The present invention provides isolated nucleic acids encoding non-human primate p40, p19, and p35 subunits useful, e.g., as reagents for expressing purified proteins of the IL-12 family of cytokines and modulating various cell types of the immune network in primates, particularly in cynomolgus macaques.

### SUMMARY OF THE INVENTION

The present invention is directed towards mammalian cytokines of the IL-12 family of cytokines. In particular, the present invention is directed towards cynomolgus macaque IL-12 cytokines.

In one embodiment, an isolated polynucleotide comprising a nucleotide sequence that encodes a cynomolgus macaque p19 polypeptide, p19 having the amino acid sequence of SEQ ID NO 2 is provided. In a particular embodiment the isolated polynucleotide encoding cynomolgus p19 comprises the nucleotide sequence of SEQ ID NO 1. In some embodiments, a nucleic acid comprising SEQ ID NO 1 or its complement is provided. In another embodiment, an isolated polynucleotide comprising a nucleotide sequence that encodes a cynomolgus macaque p40 polypeptide, p40 having the amino acid sequence of SEQ ID NO 4 is provided. In a particular embodiment the isolated polynucleotide encoding cynomolgus P40 comprises the nucleotide sequence of SEQ ID NO 3. In one embodiment, a nucleic acid comprising SEQ ID NO 3 or its complement is provided. In another embodiment, an isolated polynucleotide comprising a nucleotide sequence that encodes a cynomolgus macaque p35 polypeptide, p35 having the amino acid sequence of SEQ ID NO 6 is provided. In a particular embodiment the isolated polynucleotide encoding cynomolgus p35 comprises the nucleotide sequence of SEQ ID NO 5. In one embodiment, a nucleic acid comprising SEQ ID NO 5 or its complement is provided.

In other embodiments, an expression vector comprising a polynucleotide of the invention operably linked to an expression control sequence is provided. In one embodiment, an expression vector comprising a polynucleotide encoding a polypeptide according to SEQ ID NO 2, e.g. a polynucleotide according to SEQ ID NO 1, operably linked to an expression control sequence is provided. In another embodiment, an expression vector comprising a polynucleotide encoding a polypeptide according to SEQ ID NO 4, e.g., a polynucleotide according to SEQ ID NO 3, operably linked to an expression control sequence is provided. In another embodiment, an expression vector comprising a polynucleotide encoding a polypeptide according to SEQ ID NO 6, e.g., a polynucleotide according to SEQ ID NO 5, operably linked to an expression control sequence is provided.

In one embodiment, the polynucleotide of the invention comprises the polynucleotide sequences according to SEQ ID NOs 1 and 3 operably linked, e.g., joined by an elasti linker sequence (gttcctggagtaggggtacctggggtgggc) (SEQ ID NO 7)). In one embodiment, the 5′ end of the polynucleotide sequence according to SEQ ID NO 1 is linked by the elasti linker to the 3′ end of SEQ ID NO 3. In another embodiment, the 5′ end of the polynucleotide sequence according to SEQ ID NO 3 is linked by the elasti linker to the 3′ end of SEQ ID NO 1. In a particular embodiment, an expression vector comprising linked SEQ ED NOs 1 and 3 operably linked to an expression control sequence is provided.

In one embodiment, the polynucleotide of the invention comprises the polynucleotide sequence according to SEQ ID NOs 3 and 5 operably linked, e.g. joined by an elasti linker sequence (gttcctggagtaggggtacctggggtgggc) (SEQ ID NO 7)). In one embodiment, the 5′ end of the polynucleotide sequence according to SEQ ID NO 5 is linked by the elasti linker to the 3′ end of SEQ ID NO 3. In another embodiment, the 5′ end of the polynucleotide sequence according to SEQ ID NO 3 is linked by the elasti linker to the 3′ end of the polynucleotide sequence according to SEQ ID NO 5. In a particular embodiment, an expression vector comprising linked SEQ ID NOs 3 and 5 operably linked to an expression control sequence is provided.

In some embodiments, the invention encompasses a cell, e.g., a cultured cell, comprising an expression vector of the invention. In some embodiments, cultured cell, may be but is no limited to a prokaryotic cell, a bacterial cell, a yeast cell, an insect cell, a eukaryotic cell, a mammalian cell, a mouse cell, a primate cell, or a human cell. In some embodiments, the expression vector comprised in the cultured cell includes a polynucleotide sequence according to SEQ ID NOs 1, 3 or 5 or any combination thereof. In particular embodiments, the expression vector comprised in the cultured cell includes polynucleotides 1 and 3 or polynucleotides 3 and 5. In other embodiments, the present invention provides a tissue or organ, other than a cynomolgus tissue or organ, comprising a polynucleotide according to SEQ ID NOs 1, 3 or 5. In other embodiments, the present invention provides a tissue or organ, other than a cynomolgus tissue or organ, comprising the polynucleotides according to SEQ ID NOs 1 and 3 or the polynucleotides according to SEQ ID NOs 3 and 5.

In some embodiments, the present invention provides a method of producing a polypeptide comprising culturing a cell of the invention which comprises and expression vector of the invention under conditions permitting expression of the polypeptide. In some embodiments, the method further comprises purifying the polypeptide from the cell and/or cell medium. In some embodiments, the expression vector comprised in the cultured cell includes a polynucleotide sequence according to SEQ ID NO 1, 3 or 5 or any combination thereof. In particular embodiments, the expression vector comprised in the cultured cell includes polynucleotides 1 and 3 or polynucleotides 3 and 5. In some embodiments, the method of producing a polypeptide comprises purifying the polypeptide from a tissue or organ comprising an expression vector of the invention.

In some embodiments, the polynucleotides of the invention are immobilized to a solid support, including but not limited to a nitrocellulose filter, a bead, a multiwell plate, or a chip. In some embodiments, the immobilized polynucleotides of the invention comprise a nucleotide sequence according to SEQ ID NO 1, SEQ ID NO 3 and/or SEQ ID NO 5.

The present invention further provides an isolated or recombinantly produced polypeptide comprising an amino acid sequence according to any one of SEQ ID NOs 2, 4 or 6.

In a particular embodiment, the present invention provides a polypeptide comprising SEQ ID NOs 2 and 4 joined by an elasti linker with the amino acid sequence VPGVGVPGVG (SEQ ID NO 8). In one embodiment, the configuration of the polypeptide comprises SEQ ID NO 2—elasti linker—SEQ ID NO 4, while in another embodiment, the configuration of the polypeptide comprises SEQ ID 4—elasti linker—SEQ ID NO 2. In another embodiment, the linked polypeptide according to SEQ ID NOs 2 and 4 binds to a mammalian cell surface receptor. In a preferred embodiment, the cell surface receptor to which the linked polypeptides bind is the IL-23 receptor. In another preferred embodiment, the mammal is a primate. In a particular embodiment, the primate is a cynomolgus macaque. In a further embodiment the linked polypeptides according to SEQ ID NO 2 and 4 bind to both the human and the cynomolgus IL-23 receptor. In one embodiment, the present invention provides a composition comprising the linked polypeptide according to SEQ ID NOs 2 and 4 and a pharmaceutically acceptable carrier or diluent.

In another particular embodiment, the present invention provides a polypeptide comprising SEQ ID NOs 4 and 6 joined by an elasti linker with the amino acid sequence VPGVGVPGVG (SEQ ID NO 8). In one embodiment, the configuration of the polypeptide comprises SEQ ID NO 4—elasti linker—SEQ ID NO 6, while in another embodiment, the configuration of the polypeptide comprises SEQ ID NO 6—elasti linker—SEQ ID NO 4. In one embodiment the linked polypeptide according to SEQ ID NOs 4 and 6 binds to a mammalian cell surface receptor. In a preferred embodiment, the cell surface receptor to which the linked polypeptides bind is the IL-12 receptor. In another preferred embodiment, the mammal is a primate. In a particular embodiment, the primate is a cynomolgus macaque. In a further embodiment, the linked polypeptides according to SEQ ID NOs 4 and 6 bind to both the human and the cynomolgus IL-23 receptor. In one embodiment, the present invention provides a composition comprising the linked polypeptide comprising an amino acid sequence of SEQ ID NOs 4 and 6 and a pharmaceutically acceptable carrier or diluent.

In some embodiments, the polypeptides according to any one of SEQ ID NOs 2, 4, 6 or any combination thereof are immobilized to a solid support, including but not limited to a nitrocellulose filter, a bead, a multiwell plate, or a chip.

In one embodiment, the present invention provides a binding compound which recognizes the polypeptide according to any one of SEQ ID NOs 2, 4 or 6. In one embodiment, the binding compound modulates the activity of the polypeptide according to any one of SEQ ID NOs 2, 4, or 6. In some embodiments, the binding compound inhibits the activity of the polypeptide according to any one of SEQ ID NOs 2, 4, or 6, while in other embodiments, the binding compound stimulates the activity of polypeptide according to any one of SEQ ID NOs 2, 4, or 6. In some embodiments, the binding compound which recognizes the polypeptide according to any one of SEQ ID NOs 2, 4, or 6 is an antibody. In another embodiment, the binding compound is a small molecule. In a preferred embodiment, the binding compound is an aptamer.

In another embodiment, the present invention provides a binding compound which recognizes the linked polypeptide comprising an amino acid sequence of SEQ ID NOs 2 and 4. In one embodiment, the binding compound modulates the activity of the linked polypeptide comprising the amino acid sequence of SEQ ID NOs 2 and 4. In some embodiments, the binding compound inhibits the activity of the linked polypeptide according to SEQ ID NOs 2 and 4, while in other embodiments, the binding compound stimulates the activity of the linked polypeptide according to SEQ ID NOs 2 and 4. In some embodiments, the binding compound which recognizes the linked polypeptide according to SEQ ID NOs 2 and 4 is an antibody. In another embodiment, the binding compound is a small molecule. In a preferred embodiment, the binding compound is an aptamer.

In another embodiment, the present invention provides a binding compound which recognizes the linked polypeptide according to SEQ ID NOs 4 and 6. In one embodiment, the binding compound modulates the activity of the linked polypeptide according to SEQ ID NOs 4 and 6. In some embodiments, the binding compound inhibits the activity of the linked polypeptide according to SEQ ID NOs 4 and 6, while in other embodiments, the binding compound stimulates the activity of the linked polypeptide according to SEQ ID NOs 4 and 6. In some embodiments, the binding compound which recognizes the linked polypeptide according to SEQ ID NOs 4 and 6 is an antibody. In another embodiment, the binding compound is a small molecule. In a preferred embodiment, the binding compound is an aptamer.

In one embodiment, the present invention provides a binding compound which recognizes a nucleic acid derived from any one of the polynucleotides according to SEQ ID NOs 1, 2 or 3. In one embodiment, the binding compound modulates the expression of any one of SEQ ID NOs 1, 3, or 5. In one embodiment, the binding compound inhibits the expression of any one of SEQ ID NOs 1, 3 or 5. In some embodiments, the binding compound which recognizes any one of the polynucleotides according to SEQ ID NOs 1, 3, or 5 is selected from the group consisting of an antisense oligodeoxynucleotide or siRNA.

In some embodiments, a method for identifying a binding compound is provided. In one embodiment the identification method comprises the steps of: a) contacting a binding compound with a cynomolgus macaque polypeptide according to any one of SEQ ID NOs 2, 4, 6, SEQ ID NO 2 linked to SEQ ID NO 4 or SEQ ID NO 4 linked to SEQ ID NO 6; b) selecting the binding compound that binds to the cynomolgus macaque polypeptide to result in a candidate binding compound; c) contacting the candidate binding compound with a human polypeptide selected from the group consisting of: p19, p40, p35, IL-23 or IL-12; and d) identifying the candidate binding compound that binds to both the cynomolgus macaque polypeptide and its human homolog. In another embodiment, the identification method comprises the steps of: a) contacting a binding compound with a cynomolgus macaque polypeptide according to any one of SEQ ID NOs 2, 4, 6, SEQ ID NO 2 linked to SEQ ID NO 4, or SEQ ID NO 4 linked to SEQ ID NO 6; b) selecting the binding compound that modulates a function of the cynomolgus macaque polypeptide to result in a candidate binding compound; c) contacting the candidate binding compound with a human polypeptide selected from the group consisting of: p19, p40, p35, IL-23 or IL-12; and d) identifying the candidate binding compound that modulates the function of both the cynomolgus macaque polypeptide and its human homolog. In some embodiments, the identification method comprises using a polypeptide of the invention, e.g. a polypeptide according to any one of SEQ ID NOs 2, 4, 6, SEQ ID NO 2 linked to SEQ ID NO 4, or SEQ ID NO 4 linked to SEQ ID NO 6, in SELEX™ to result in an aptamer to a polypeptide of the invention. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”.

In some embodiments, the identification method of the invention comprises administering a polypeptide of the invention to an animal e.g. a polypeptide according to any one of SEQ ID NOs 2, 4, 6, SEQ ID NO 2 linked to SEQ ID NO 4, or SEQ D NO 4 lined to SEQ ID NO 6, to result in an antibody to a polypeptide of the invention.

### BRIEF DESCRIPTION OF THE DRAWINGS

FIG. 1A shows an alignment of the mature peptide sequence of cynomolgus p40 of the present invention to human p40; FIG. 1B shows an alignment of the mature peptide sequence of cynomolgus p19 of the present invention to human p19; FIG. 1C shows an alignment of the mature peptide sequence of cynomolgus p35 of the present invention to the human p35.

FIG. 2A shows the binding of cynomolgus IL-23 of the present invention to the human IL-23R receptor measured by ELISA; FIG. 2B shows the binding of cynomolgus IL-12 of the present invention to the human IL-12RB1 receptor subunit measured by ELISA.

FIG. 3 shows a comparison of dot blot binding curves (in duplicate) of the human IL-23 aptamer, ARC1623, to human IL-23, cynomolgus IL-23 and cynomolgus IL-12.

FIG. 4 shows a comparison of dot blot binding curves (in duplicate) of the IL-23 aptamer, ARC1626, to human IL-23, cynomolgus IL-23 and cynomolgus IL-12.

FIG. 5 is a graph showing the absorbance (vertical axis) of decreasing concentrations of cyno IL-23 and a human IL23 control in an Elisa assay.

### DETAILED DESCRIPTION OF THE INVENTION

The details of one or more embodiments of the invention are set forth in the accompanying description below. Although any methods and materials similar or equivalent to those described herein can be used in the practice or testing of the present invention, preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control. All references cited herein are incorporated in their entirety.

The present invention provides polypeptides which encode gene members of the cynomolgus macaque IL-12 family of cytokines including p40, p19, and p35. As used herein the term “polypeptide” refers to any chain of amino acids linked by peptide bonds and is synonymous with “protein” (e.g., cytokines), however the term polypeptide as used herein is not restricted to structures similar to those produced by organisms. In one embodiment, the polypeptides of the present invention are mature peptides. As used herein, a mature peptide refers to coding sequence for the mature or final peptide or protein product following post-translational modification. The present invention also provides polynucleotides encoding polypeptides for cynomolgus IL-23 and IL-12. As used herein, the term “polynucleotide” refers to a polymer of nucleotides bonded to one another by phosphodiester bonds, and includes nucleic acids such as DNA or cDNA. The polynucleotides provided by the present invention may be synthesized by standard methods.

Also provided are expression vectors comprising the isolated polynucleotides aforementioned, useful as reagents for expressing and purifying cynomolgus IL-23 and IL-12 cytokines, i.e. proteins. The term expression vector as used herein refers to the integration of cDNA isolated from a donor source into a plasmid capable of directing synthesis of the protein encoded by the cDNA. The term host as used herein refers to an in vitro cell culture, including but not limited to prokaryotic cells, bacterial cells, yeast cells, insect cells, eukaryotic cells, mammalian cells, mouse cells, primate cells, and human cells. The term purified polypeptide as used herein encompasses protein that is substantially free from other contaminating proteins, nucleic acids, or other biologics derived from the donor source. Purity of the polypeptide may be measured by SDS-PAGE gel analysis, and is usually at least 70%, preferably at least 80%, more preferably at least 90%, and more preferably at least 95% pure as measured by SDS-PAGE gel. These purified cytokines can be used as targets for identifying or screening for binding compounds which may modulate the function of such polypeptides.

Compounds that Bind to Cynomolgus and Human IL-23 and IL-12 Cytokines

The purified cynomolgus IL-23 and IL-12 cytokines provided by the present invention can be used to identify, screen for or generate compounds which recognize and bind to IL-23 and/or IL-12. The present invention encompasses compounds which recognize and bind cynomolgus IL-23 and/or IL-12 cytokines and modulate their activity. The present invention also encompasses compounds which recognize and bind to cynomolgus IL-23 and/or IL-12 and cross react with human IL-23 and/or IL-12 cytokines and modulate the activity of both cynomolgus and human cytokines. The present invention additionally encompasses compounds which recognize and bind to human IL-23 and/or IL-12 and cross react with cynomolgus IL-23 and/or IL-12 and modulate the activity of both human and cynomolgus cytokines. In some embodiments, the binding compounds stimulate cytokine activity, while in other embodiments, the compounds inhibit cytokine activity. Compounds capable of binding and modulating the activity of cynomolgus and/or human IL-23 and/or IL-12 cytokines which are encompassed by the present invention include but are not limited to aptamers, antibodies, and small molecules.

The present invention also encompasses nucleic acid binding compounds including antisense oligonucleotides, and, siRNA. Such binding compounds may be useful as therapeutic, research and/or diagnostic tools.

The present invention also encompasses compositions comprising the binding compounds or nucleic acid binding compounds encompassed by the present invention and a pharmaceutically acceptable carrier or diluent. These compositions may also be useful as therapeutics for abnormal inflammatory or autoimmune conditions in primates when administered in a pharmaceutically effective amount. Such compounds may be administered to primates alone or in combination with other known treatments.

### EXAMPLES

### Example 1

### Polynucleotides Encoding Cynomolgus Macaque Polypeptides of the IL-12 Family of Cytokines

As previously described, the IL-12 family of cytokines contains a variety of heterodimeric molecules which have the p40 subunit in common, including IL-23 and IL-12. IL23 is composed of p40 and p19 subunits while IL-12 is composed of p40 and p35 subunits. Non-human primate cDNAs of all three subunits were isolated from Cynomolgus macaque normal spleen cDNA (Biochain Institute, Inc., Hayward, Calif.) using a two-step PCR method. As used herein, the term cDNA refers to a single strand of DNA synthesized in a lab to complement the bases in a given strand of messenger RNA, which represents the parts of a gene that are expressed in a cell to produce a protein. To isolate the p40 subunit, the following oligos were used with the cynomolgus normal spleen cDNA template for the first PCR amplification step under the following cycling conditions: 95° C., 30 s; 50° C., 30 s; and 72° C. 60 s, for 35 cycles.

(SEQ ID NO 11)

| | |
|---|---|
| 5′ oligo | 5′-AGATGTGTCACCAGCAG(T/G)TGGTCATCTCTTGG |

(SEQ ID NO 12)

| | |
|---|---|
| 3′ oligo | 5′-GTTTTGCTTTAATATCTTCTACTTTTCCTCC |

The first round PCR amplified a 1042 bp fragment which was used as a template for the second PCR step. The second PCR step was performed using the following oligos under the cycling conditions: 95° C., 30 s; 57.5° C., 30 s; and 72° C. 60 s, for 25 cycles.

(SEQ ID NO 13)

| | |
|---|---|
| 5′ oligo | 5′-GGTTTTCCCTGGTTTTTCTGGCATCTCCCCT |

(SEQ ID NO 14)

| | |
|---|---|
| 3′ oligo | 5′-TCCTGGATCAGAACCTAACTGCAGGGC |

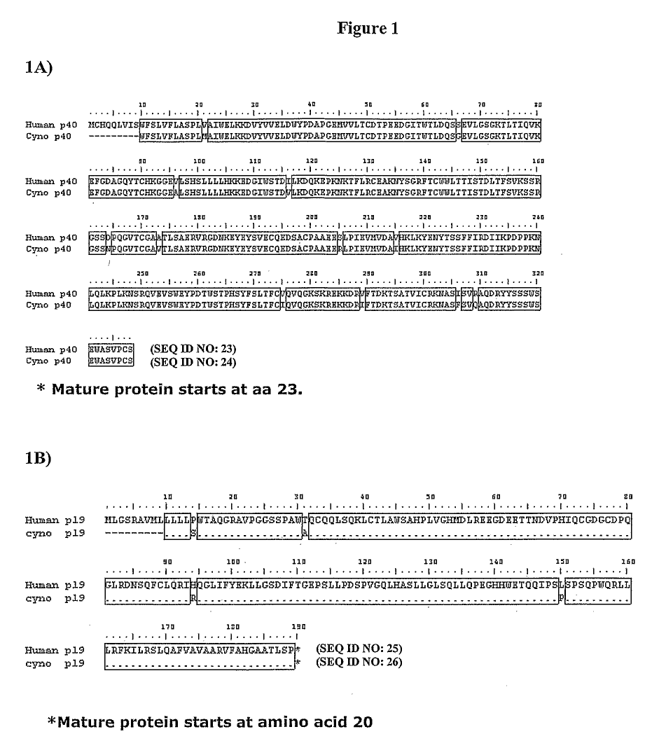

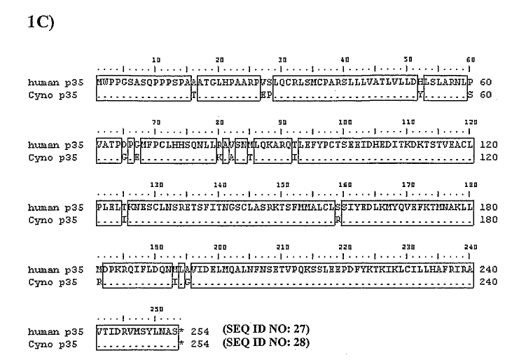

This amplified a 944 bp fragment which was cloned into a TA cloning vector (pCR2.1TOPO, E. coli strain Top10, Invitrogen, Carlsbad, Calif.) following the manufacturer's protocol. Plasmid DNA was prepared from 1.5 mL of bacterial culture using QIAprep kit (QIAGEN, Hilden, Germany) and sequenced. The resulting DNA sequence (SEQ ID NO 3) isolated from cynomolgus normal spleen cDNA, and the corresponding mature peptide sequence (SEQ ID NO 4), are listed in Table 1 below. The mature peptide sequence encoded by the isolated nucleic acid sequence of cynomolgus p40 (SEQ ID NO 4), and a comparison of the alignment with human p40 is shown in FIG. 1.

To isolate the p19 subunit the following oligos were used with the cynomolgus normal spleen cDNA template for a first PCR amplification step under the following cycling conditions: 95° C., 30 s; 50° C., 30 s; and 72° C. 60 s, for 35 cycles.

(SEQ ID NO 15)

| | |
|---|---|
| 5′ oligo | 5′-AGATTTGAGAAGAAGGCAAAAAGATG |

(SEQ ID NO 16)

| | |
|---|---|
| 3′ oligo | 5′-TCTGAGTGCCATCCTTGAGCTAATGGCTTTA |

The first round PCR amplified a 624 bp fragment which was used as a template for a second PCR step. The second PCR step was performed using the following oligos under the cycling conditions: 95° C., 30 s; 57.5° C., 30 s; and 72° C. 60 s, for 25 cycles.

(SEQ ID NO 17)

| | |
|---|---|
| 5′ oligo | 5′-GCTGTTGCTGCTGTCCTGGACAGCTCAGGGC |

(SEQ ID NO 18)

| | |
|---|---|
| 3′ oligo | 5′-AGCTGCTGCCTTTAGGGACTCAGGGTTGC |

This amplified a 566 bp fragment which was cloned into a TA cloning vector (pCR2.1TOPO, E. coli strain Top10, Invitrogen, Carlsbad, Calif.) following the manufacturer's protocol. Plasmid DNA was prepared from 1.5 mL of bacterial culture using QIAprep kit (QIAGEN, Hilden, Germany) and sequenced. The resulting DNA sequence (SEQ ID NO 1) isolated from cynomolgus normal spleen cDNA, and the corresponding mature peptide sequence (SEQ ID NO 2), are listed in Table 1 below. The mature peptide sequence encoded by the isolated nucleic acid sequence of cynomolgus p19 (SEQ ID NO 2), and a comparison of the alignment with human p19 is shown in FIG. 1.

To isolate the p35 subunit the following oligos were used with the cynomolgus normal spleen cDNA template for a first PCR amplification step under the following cycling conditions: 95° C., 30 s; 50° C., 30 s; and 72° C. 60 s, for 35 cycles.

(SEQ D NO 19)

| | |
|---|---|
| 5′ oligo | 5′-TCGGGACAATTATAAAAATGTGGC |

(SEQ ID NO 20)

| | |
|---|---|
| 3′ oligo | 5′-CCTCGCITTTTAGGAAGCATTC |

The first round PCR amplified a 788 bp fragment which was used as a template for a second PCR step. The second PCR step was performed using the following oligos under the cycling conditions: 95° C., 30 s; 57.5° C., 30 s; and 72° C. 60 s, for 25 cycles.

(SEQ ID NO 21)

| | |
|---|---|
| 5′ oligo | 5′-AAAATGTGGCCCCCTGGGTCAGGCT |

(SEQ ID NO 22)

| | |
|---|---|
| 3′ oligo | 5′-TTTTAGGAAGCATTCAGATAGC |

This amplified a 767 bp fragment which was cloned into a TA cloning vector (pCR2.1TOPO, E. coli strain Top10, Invitrogen, Carlsbad, Calif.) following the manufacturer's protocol. Plasmid DNA was prepared from 1.5 mL of bacterial culture using QIAprep kit (QIAGEN, Hilden, Germany) and sequenced. The resulting DNA sequence (SEQ ID NO 5) isolated from cynomolgus normal spleen cDNA, and the corresponding mature peptide sequence (SEQ ID NO 6), are listed in Table 1 below. The mature peptide sequence encoded by the isolated nucleic acid sequence of cynomolgus p35 (SEQ ID NO 6), and a comparison of the alignment with human p35 is shown in FIG. 1.

TABLE 1

Nucleic Acid Sequences Encoding Mature Cynomolgus p40, p19, and p35 genes
and the Corresponding Mature Peptide Sequences

p19: (SEQ ID NO 1)
AGGGCTGTGCCTGGGGGCAGCAGCCCTGCCTGGGCTCAGTGCCAGCAGCTTTCACAGAAGCTCTGCACACTGGCCTGGAGTGCA
CATCCACTAGTGGGACACATGGATCTAAGAGAAGAGGGAGATGAAGAGACTACAAATGATGTTCCCCATATCCAGTGTGGAGA
TGGCTGTGACCCCCAAGGACTCAGGGACAACAGTCAGTTCTGCTTGCAAAGGATTCGCCAGGGTCTGATTTTTTACGAGAAGCT
ACTGGGATCGGATATTTTCACAGGGGAGCCTTCTCTGCTGCCTGATAGCCCTGTGGGCCAGCTTCATGCCTCCCTACTGGGCCTC
AGCCAACTCCTGCAGCCTGAGGGTCACCACTGGGAGACTCAGCAGATTCCAAGCCCCAGTCCCAGCCAGCCATGGCAGCGCCT
CCTTCTCCGCTTCAAAATCCTTCGCAGCCTCCAGGCCTTTGTGGCTGTAGCTGCCCGGGTCTTTGCCCATGGAGCAGCAACCCTG
AGTCCCTAA
p19: (SEQ ID NO 2)
RAVPGGSSPAWAQCQQLSQKLCTLAWSAHPLVGHMDLREEGDEETTNDVPHIQCGDGCDPQGLRDNSQFCLQRIRQGUFYEKLLGS
DIFTGEPSLLPDSPVGQLHASLLGLSQLLQPEGHHWETQQIPSPSPSQPWQRLLLRFKILRSLQAFVAVAARVFAHGAATLSP
p40: (SEQ ID NO 3)
ATATGGGAACTGAAGAAAGACGTTTATGTTGTAGAATTGGACTGGTACCCGGATCCCCCTGGAGAAATGGTGGTCCTCACCTGT
GACACCCCTGAAGAAGATGGTATCACCTGGACCTTGGACCAGAGTGGTGAGGTCTTAGGCTCTGGCAAAACCCTGACCATCCA
AGTCAAAGAGTTTGGAGATGCTGGCCAGTACACCTGTCACAAAGGAGGCGAGGCTCTAAGCCATTCACTCCTGCTGCTTCACAA
AAAGGAAGATGGAATTTGGTCCACTGATGTTTTAAAGGACCAGAAAGAACCCAAAAATAAGACCTTTCTAAGATGCGAGGCCA
AAAATTATTCTGGACGTTTCACCTGCTGGTGGCTGACGACAATCAGTACTGATCTGACATTCAGTGTCAAAAGCAGCAGAGGCT
CTTCTAACCCCCAAGGGGTGACGTGTGGAGCCGTTACACTCTCTGCAGAGAGGGTCAGAGGGGACAATAAGGAGTATGAGTAC
TCAGTGGAGTGCCAGGAGGACAGTGCCTGCCCAGCCGCTGAGGAGAGGCTGCCCATTGAGGTCATGGTGGATGCCATTCACAA
GCTCAAGTATGAAACTACACCAGCAGCTTCTrCATCAGGGACATCATCAAACCCGACCCACCCAAGAACTTGCAGCTGAAGCC
ATTAAAGAATTCTCGGCAGGTTGAGGTCAGCTGGGAGTACCCTGACACCTGGAGTACTCCACATTCCTACTTCTCCCTGACATTC
TGCATCCAGGTCCAGGGCAAGAGCAAGAGAGAAAAGAAGATAGAATCTTCACAGACAAGACCTCAGCCACGGTCATCTGCCG
CAAAAATGCCAGCTTTAG
p40: (SEQ ID NO 4)
IWELKKDVYVVELDWYPDAPGEMVVLTCDTPEEDGITWTLDQSGEVLGSGKTLTIQVKEFGDAGQYTCHKGGEALSHSLLLLHKKE
DGIWSTDVLKDQKEPKNKTFLRCEAKNYSGRFTCWWLTTISTDLTFSVKSSRGSSNPQGVTCGAVTLSAERVRGDNKEYEYSVECQE
DSACPAAEERLPIEVMVDAIHKLKYENYTSSFFIRDIIKPDPPKNLQLKPLKNSRQVEVSEYPDTWSTPHSYFSLTFCIQVQGKSKREK
KDRTFTDKTSATVICRKNASFSVQAQDRYYSSSWSEWASVPCS
p35: (SEQ ID NO 5)
AGAAACCTCTCCGTGGCCACCCCAGGCCCAGAAATGTTCCCGTGCCTTCACCACTCCCAAAACCTGCTGAAGGCCGCCAGCAAC
ACGCTTCAGAAGGCCAGACAAATTCTAGAATTTTACCCTTGCACTTCTGAAGAGATTGATCATGAAGATATCACAAAAGATAAA
ACCAGCACAGTAGAGGCCTGTTTACCATTGGAATTAATCAAGAATGAGAGTTGCCTAAATTCCAGAGAGACTTCTTTCATAACT
AATGGGAGTTGCCTGGCCTCCAGAAAGACCTCTTTTATGATGGCCCTGTGCCTTAGGAGTATTTATGAAGACTTGAAGATGTAC
CAAGTGGAGTTCAAGACCATGAATGCAAAGCTTCTGAGGGATCCTAAGAGGCAGATCTTTCTAGATCAAAACATACTGGGAGT
TATGATGAGCTGATGCAGGCCCTGAATTTCAACAGTGAGACTGTGCCACAAAAATCCTCCCTTGAAGAACCGGATTTTTATAA
AACTAAAATCAAGCTCTGCATACTTCTTCATGCTTTCAGAATTCGGGCAGTGACTATTGATAGAGTGATGAGCTATCTGAATGCT
TCCTAAAAAAGGGC
p35 (SEQ ID NO 6)
RNLSVATPGPEMFPCLHHSQNLLKAASNTLQKARQILEFYPCTSEEIDHEDITKDKTSTVEACLPLELIKNESCLNSRETSFJTNGSCLAS
RKTSFMMALCLRSIYEDLKMYQVEFKTMNAKLLRDPKRQIFLDQNILGVIDELMQALNFNSETVPQKSSLEEPDFYKTKIKLCILLHAF
RIRAVTIDRVMSYLNAS

### Example 2

### Expression and Purification of Cynomolgus IL-23 and IL-12 Cytokines

The present invention additionally provides expression constructs comprising the polynucleotide sequences in Table 1 useful as reagents for expressing the cDNA in a host source and isolating purified protein encoded by the cDNA. As previously described, IL-23 is a heterodimer composed of p19 and p40 subunits, encoded by two separate genes. A cynomolgus IL-23 expression construct was made by combining both the p40 (SEQ ID NO 3) cDNA and p19 (SEQ ID NO 1) cDNA isolated from cynomolgus normal spleen into a single chain linked with 2 bovine elastin motifs (gttcctggagtaggggtacctggggtgggc, SEQ ID NO 7, which encompasses 2 elasti motifs)) in the following order: SEQ ID NO 3-SEQ ID NO 7-SEQ ID NO 1. This single chain was integrated into pORF plasmid (Invivogen, San Diego, Calif.) using conventional molecular cloning techniques as follows. The cyno p40 fragment was PCR amplified to replace human p40 using BspeI and ApaI restriction sites and the cyno p19 fragment was PCR amplified to replace human p19 using Acc65I and NheI restriction sites in the pORF-hIL23 vector (Invivogene, San Diego, Calif.). pORF-hIL23 is a human IL-23 expression vector containing both human p40 and human p19 in a single chain linked with 2 bovine elastin motifs (VPGVGVPGVG). The resulting plasmid contains both cyno p40 and cyno p19 in a single chain linked with 2 bovine elastin motifs.

The cynomolgus. IL-23 expression construct was transfected into human Free-style 293F cells (Invitrogen, Carlsbad, Calif.) using a standard transfection method. Three days post transfection, cells were centrifuged at 1000 rpm for 5 minutes and the supernatant was collected. After adding a mini-complete protease inhibitor tablet (Roche Diagnostics, Germany), the supernatant was cooled on ice for 30 minutes and then subjected to dialysis against Buffer A (20 mM Tris-Cl, pH 8.0, 20 mM NaCl) for 4 hours, changing Buffer A once halfway through dialysis. After dialysis, 30 mL of supernatant was applied to a MonoQ 5/5 ion exchange column (Amersham Biosciences, Piscataway, N.J.) using an AKTA Explore system (Amershain Biosciences, Piscataway, N.J.). The column was washed with 8 mL of 0.05 M NaCl in Buffer A. Bound proteins were eluted with a 20 mL gradient of 0.05 M to 1 M NaCl in Buffer A.

IL-12 is a heterodimer composed of p35 and p40 subunits, encoded by two separate genes. A cynomolgus IL-12 expression construct was made by combining both the p40 (SEQ ID NO 3) and p35 (SEQ ID NO 5) cDNA isolated from cynomolgus normal spleen into a single chain linked with 2 bovine elastin motifs (gttcctggagtaggggtacctggggtgggc, SEQ ID NO 7) in the following order: SEQ ID NO 3-SEQ ID NO 7-SEQ ID NO 5. This single chain was integrated into pORF plasmid (Invivogene, San Diego, Calif.) using conventional molecular cloning techniques as follows: The cyno p40 fragment was PCR amplified to replace human p40 using BspeI and ApaI restriction sites and the cyno p35 fragment was PCR amplified to replace human p19 using Acc65I and NheI restriction sites in the pORF-hIL23 vector (Invivogene, San Diego, Calif.). The resulting plasmid contains both cyno p40 and cyno p35 in a single chain linked with 2 bovine elastin motifs.

The cynomolgus IL-12 expression construct was transfected into human Free-style 293F cells (Invitrogen, Carlsbad, Calif.). Three days post transfection, cells were centrifuged at 1000 rpm for 5 minutes and the supernatant was collected. After adding a mini-complete protease inhibitor tablet (Roche Diagnostics, Germany), the supernatant was cooled on ice for 30 minutes and then subjected to dialysis against Buffer A (20 mM Tris-Cl, pH 8.0, 20 mM NaCl) for 4 hours, changing Buffer A once halfway through dialysis. After dialysis, 30 mL of supernatant was applied to a MonoQ 515 ion exchange column (Amersham Biosciences, Piscataway, N.J.) using an AKTA Explore system (Amersham Biosciences, Piscataway, N.J.). The column was washed with 8 mL of 0.05 M NaCl in Buffer A. Bound proteins were eluted with a 20 mL gradient of 0.05 M to 1 M NaCl in Buffer A.

Both cynomolgus 123 and IL-12 purified proteins were analyzed by SDS-PAGE gel. Cynomolgus IL-23 ran at approximately 69 kD and was approximately 80% pure, while cynomolgus IL-12 ran between 66-80 kD and was approximately 95% pure. The purified IL-23 and IL-12 were then each pooled and aliquoted, and stored at −80° C.

The present invention also provides compositions comprising the cynomolgus IL-23 and IL-12 purified proteins and a pharmaceutically acceptable carrier solution or diluent. Such compositions have commercial potential and will be useful for diagnostic, therapeutic, and research purposes. While not intending to be bound by theory, these compositions may be useful for modulating the immune system in primates. In particular, the compositions provided by the present invention may be useful for the diagnosis or treatment of inflammatory and autoimmune related diseases in primates.

### Example 3

### Cross-Species Reactivity of Cynomolgus IL-23 and IL-12

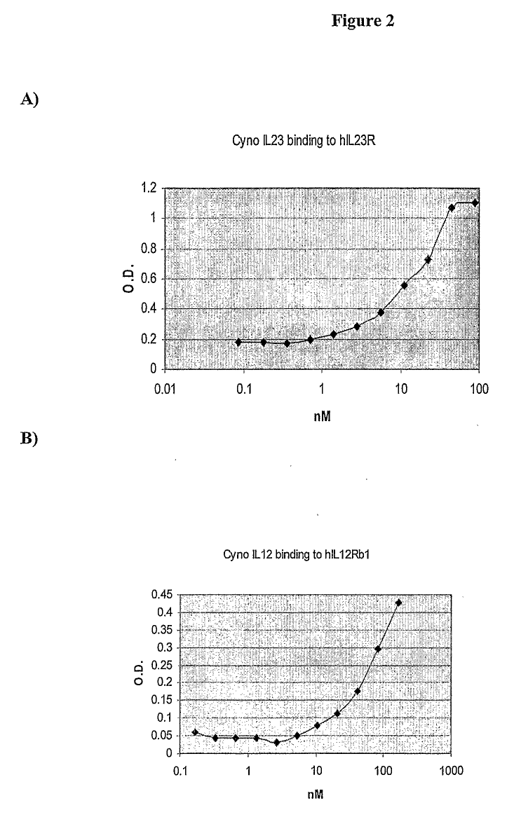

To test whether cynomolgus IL-23 binds to the human IL23 receptor (“IL23R”), and whether cynomolgus IL-12 binds to the human IL12 receptor (“IL12RB1”), human IL23R Fc and human IL12RB1 Fc fusion proteins were purchased (R & D systems) for an ELISA assay. To capture IL23R Fc fusion protein, 500 ng of IL23R—Fc protein in 100 μl of PBS (pH 7.4) was put onto a 96-well Maxisorb plate (NUNC, Rochester, N.Y.) and incubated overnight at 4° C. Likewise, to capture IL12RB1 Fc fusion protein, 500 ng of IL12RB1 Fe protein in 100 μl of PBS (pH 7.4) was put onto a 96-well Maxisorb plate (NUNC, Rochester, N.Y.) and incubated overnight at 4° C. For each, the capture solution was thrown away after overnight incubation and the plate was washed with 200 μl per well of TBST (25 mM Tris-HCl pH 7.5, 150 mM NaCl and 0.01% Tween 20) three times. The plate was then blocked with 200 μl per well of TBST containing 5% nonfat dry milk for 30 minutes at room temperature. After blocking, the plate was washed 3 times with 200 μl per well of TBST at room temperature and a titration of cynoIL23 or cynoIL12 in PBS was added to the plate and incubated at room temperature for 1 hour. The plate was then washed 3 times with 200 μl per well of TBST and 100 μl per well of two anti-human p40 monoclonal antibody from R & D systems (1:1000 each) was added and incubated for 1 hour at room temperature. After washing three times with 200 μl per well of TBST, 100 μl of HRP linked goat-anti-mouse antibody (Cell signaling, MA)) was added to each well and incubated at room temperature for 0.5 hours. Then, the plate was washed three times with 200 μl per well of TBST and 100 μl per well of TMP solution Pierce) was added and incubated in the dark at room temp for 5 minutes. A 100 μl solution containing 2 NH₂SO₄ was added to stop the reaction and the plate was read on a 96 well SpectroMax plate reader at 450 nm. FIG. 2 shows the binding curves of cynomolgus IL-23 and IL-12 of the present invention to the human IL-23 and human IL-12RB1 receptor subunits, indicating that cynomolgus IL-12 and IL-23 cross react and are capable of binding to the corresponding human receptors.

### Example 4

### Cross Species Reactivity of Binding Compounds to IL-23 and IL-12

Two aptamers previously identified through the SELEX™ process, ARC1623 and ARC1626, which bind with high affinity to human IL-23 (˜0.2 nM and ˜0.1 nM respectively as measured by dot blot analysis), were tested for their ability to recognize and bind to cynomolgus IL-23. Additionally, given that IL-23 and IL-12 have the p40 subunit in common, both human IL-23 aptamers were tested for their ability to recognize and bind to cynomolgus IL-12. The sequences for ARC1623 and ARC1626 are listed (in the 5′ to 3′ direction) in Table 2 below, where “d” denotes deoxy nucleotides, “m” denotes 2′-OMe nucleotides, “s” denotes a phosphorothioate internucleotide linkage, and “3T” denotes a 3′ inverted deoxy thymidine:

TABLE 2
Nucleic acid sequences of human IL-23 aptamers

ARC1623

(SEQ ID NO 9)

dAmCdAdGdGmCdAdAdGmUdAdAmUmUdGmGmG-s-dG-s-dA-s-
dGmU-s-dGmCmGmGdGmCdGdGmGmGmUdGmU-3T
ARC 1626

(SEQ ID NO 10)

dAmC-s-dA-s-dG-s-dGmC-s-dA-s-dA-s-dGmU-s-dA-s-
dAmUmU-s-dGmGmG-s-dG-s-dA-s-dGmU-s-dGmCmGmG-s-
dGmC-s-dG-s-dGmGmGmU-s-dGmU-3T

Aptamer binding affinity determination. Trace 5′-³²P-labeled aptamers were combined with protein (IL-23 or IL-12) at various concentrations and incubated at room temperature for 30 minutes in Dulbecco's PBS (PBS plus Ca²⁺, Mg²⁺) plus 0.1 mg/mL BSA. Each titration curve was tested in duplicate. The reactions were then added to a dot blot apparatus (Minifold-1 Dot Blot, Acrylic, Schleicher and Schuell, Keene, N.H.), assembled (from top to bottom) with Protran nitrocellulose (Schleicher and Schuell, Keene, N.H.), Hybond-P nylon (Amersham Biosciences, Piscataway, N.J.) and GB002 gel blot paper (Schleicher and Schuell, Keene, NH). Aptamers bound to protein are retained on the nitrocellulose filter, whereas the non-protein bound RNA is captured on the nylon filter. The extent of aptamer capture on each filter was quantitated using a Phosphoimager (Molecular Dynamics). Equilibrium dissociation constants (K_{D}) were calculated using the equation:

((A+P+K)−sqrt((A+P+K)̂2−4*A*P))/2A+B; where A=[aptamer]ₜₒₜₐₗ, P=[protein]ₜₒₜₐₗ and B=background signal.

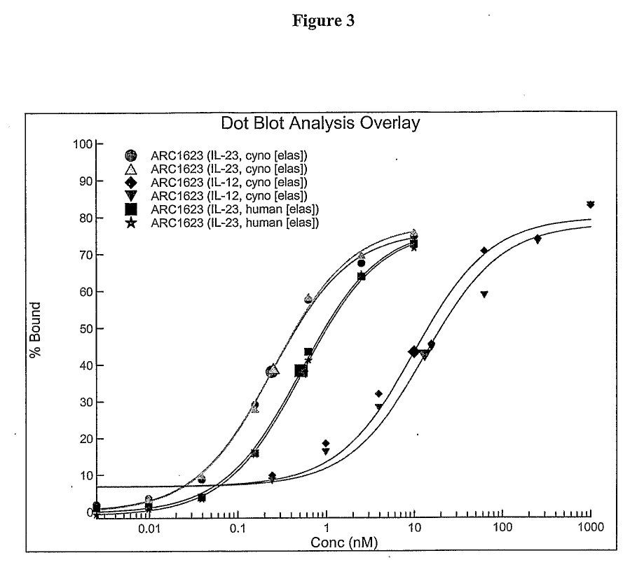

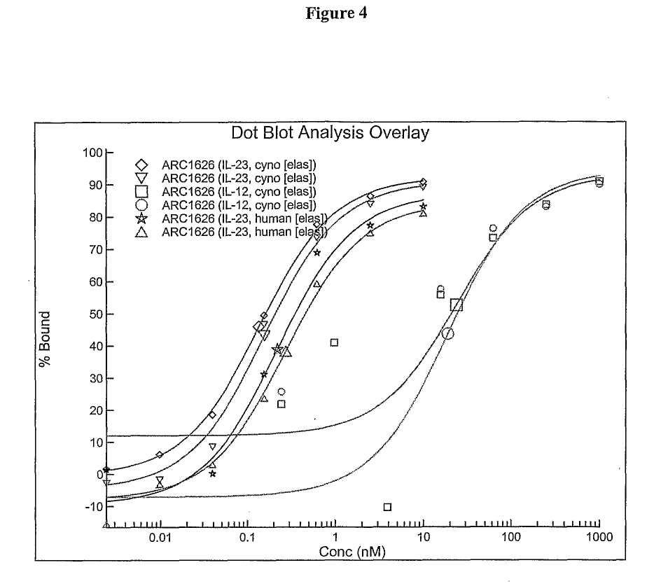

FIGS. 3 and 4 show the comparison of the aptamer binding curves (in duplicate) to cynomolgus IL-23, cynomolgus IL-12, and human IL-23. Table 3 shows the average calculated K_{D} values for the binding curves. As can be seen from FIGS. 3 and 4 and the calculated K_{D} values listed in Table 3 below, both human IL-23 aptamers cross react with cynomolgus IL-23, and bind with relatively high affinity to cynomolgus IL-23. Both human aptamers also cross react with cynomolgus IL-12, although with significantly weaker binding affinity than to human or cynomolgus IL-23.

TABLE 3
Binding affinity of human IL-23 aptamers
to human and cynomolgus IL-23 and IL-12

| | K_{D} Human IL-23 | K_{D} Cyno IL-23 | K_{D} Cyno IL-12 |
|---|---|---|---|
| Aptamer ID | (nM) | (nM) | (nM) |
| ARC1623 | 0.25 | 0.52 | 12 |
| ARC1626 | 0.15 | 0.25 | 22 |

### Example 5

### Cynomolgus IL23 STAT-3 Phosphorylation Activity

IL-23 plays a role in JAK/STAT signal transduction and phosphorylates STAT 1, 3, 4, and 5. To test whether Cynomolgus IL-23 has cell-based activity, signal transduction was assayed in the lysates of peripheral blood mononuclear cells (PBMCs) grown in media containing PHA (Phytohemagglutinin), or PHA Blasts. More specifically, the cell-based assay measuring STAT-3 phosphorylation in PHA Blasts is used determine the Cynomolgus IL-23 activity.

In essence, lysates of IL-23 treated cells will contain more activated STAT3 than quiescent cells. Stimulation of STAT3 phosphorylation was measured by the PathScan® Phospho-Stat3 (Tyr705) Sandwich ELISA Kit (Cell Signaling Technologies, Beverly, Mass.). CST's Pathscan® Phospho-Stat3 (Tyr705) Sandwich ELISA Kit is a solid phase sandwich enzyme-linked immunosorbent assay (ELISA) that detects endogenous levels of Phospho-Stat3 (Tyr705) protein. A Stat3 rabbit monoclonal antibody (#7300) has been coated onto the microwells. After incubation with cell lysates, both nonphospho- and phospho-Stat3 proteins are captured by the coated antibody. Following extensive washing, a phospho-Stat3 mouse monoclonal antibody #9138 is added to detect the captured phospho-Stat3 protein. HRP-linked anti-mouse antibody #7076 is then used to recognize the bound detection antibody. HRP substrate, TMB, is added to develop color. The magnitude of optical density for this developed color is proportional to the quantity of phospho-Stat3 protein.

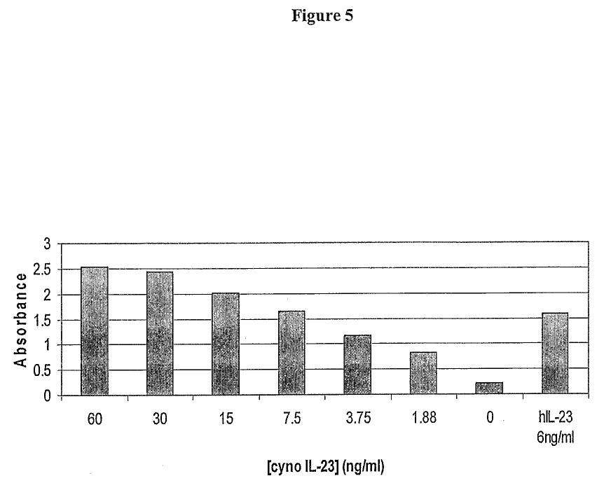

The cell-based assay was conducted by isolating the peripheral blood mononuclear cells (PBMCs) from human whole blood using a Histopaque gradient (Sigma, St. Louis, Mo.). The PBMCs were cultured for 5 to 6 days at 37° C./5% CO₂ in Peripheral Blood Medium (Sigma, St. Louis, Mo.) which contains PHA, supplemented with IL-2 (5 ug/mL) (R&D Systems, Minneapolis, Minn.), to generate PHA Blasts. PHA Blasts were washed twice with 1×PBS, then serum starved for four hours in RPMI, 0.20% FBS. After serum starvation, approximately 2.5×10⁵ cells were aliquotted into appropriately labeled eppendorf tubes. Various concentrations of Cynomolgus IL-23 were added to the aliquotted cells in a final volume of 100 μl and incubated at 37° C. for 15 minutes. As a position control, 6 ng/ml of human IL-23 (R&D Systems, Minneapolis, Minn.) was also used in the assay. The incubation reaction was stopped by adding 0.8 mL of ice-cold PBS with 1.5 mM Na₃VO₄. Cell lysates were made using the lysis buffer provided by the ELISA assay (Cell Signaling Technologies, Beverly, Mass.) following the manufacturer's instructions as mentioned above. Our results (FIG. 5) demonstrated that the recombinant purified Cynomolgus IL-23 stimulates STAT3 phosphorylation in a dose-dependent fashion. Also the specific activity of Cynomolgus IL-23 in stimulation of STAT3 phosphorylation is comparable to that of human IL-23.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

The invention having now been described by way of written description and example, those of skill in the art will recognize that the invention can be practiced in a variety of embodiments and that the description and examples above are for purposes of illustration and not limitation of the following claims.

1) An isolated polynucleotide comprising a sequence selected from a sequence that encodes a polypeptide with the amino acid sequence of SEQ ID NO 2; a sequence that encodes a polypeptide with the amino acid sequence of SEQ ID NO 4; and a sequence that encodes a polypeptide with the amino acid sequence of SEQ ID NO 6.

2) The isolated polynucleotide of claim 1, comprising a sequence selected from the nucleotide sequence of SEQ ID NO 1, the nucleotide sequence of SEQ ID NO 3; and the nucleotide sequence of SEQ ID NO 5.

3) (canceled)

4) (canceled)

5) (canceled)

6) (canceled)

7) An expression vector comprising polynucleotide of claim 1 operably linked to an expression control sequence.

8) A cultured cell comprising the vector of claim 7.

9) A method of producing a polypeptide, the method comprising culturing the cell of claim 8 under conditions permitting expression of the polypeptide.

10) The method of claim 9, further comprising purifying the polypeptide from the cell or the cell medium.

11) An expression vector comprising the polynucleotide of claim 3 on operably linked to an expression control sequence.

12) A cultured cell comprising the vector of claim 11.

13) A method of producing a polypeptide, the method comprising culturing the cell of claim 12 under conditions permitting expression of the polypeptide.

14) The method of claim 13, further comprising purifying the polypeptide from the cell or the cell medium.

15) An expression vector comprising the polynucleotide of claim 5 operably linked to an expression control sequence.

16) A cultured cell comprising the vector of claim 15.

17) A method of producing a polypeptide, the method comprising culturing the cell of claim 16 under condition permitting expression of the polypeptide.

18) The method of claim 17, further comprising purifying the polypeptide from the cell or cell medium.

19) An expression vector comprising the polynucleotide of SEQ ID NO 3 operably linked to an expression control sequence and a second polynucleotide operably linked to SEQ ID NO 3, wherein the second polynucleotide comprises a sequence selected from SEQ ID NO 1 and SEQ ID NO 5.

20) The expression vector of claim 19, wherein the second polynucleotide comprises SEQ ID NO 1, and wherein SEQ ID NO 3 is operably linked to SEQ ID NO 1 via SEQ ID NO 7.

21) A cultured cell comprising the vector of claim 19.

22) A method of producing a polypeptide, the method comprising culturing the cell of claim 21 under conditions permitting the expression of the polypeptide.

23) (canceled)

24) The expression vector of claim 23, wherein the second polynucleotide comprises SEQ ID NO 5, and wherein SEQ ID NO 3 is operably linked to SEQ ID NO 5 via SEQ ID NO 7.

25) (canceled)

26) (canceled)

27) An isolated polypeptide comprising a sequence selected from the amino acid sequence of SEQ ID NO 2, the amino acid sequence of SEQ ID NO 4, the amino acid sequence of SEQ ID NO 6, an amino acid sequence comprising SEQ ID NOs 2 and 4 joined by a synthetic amino acid linker, and an amino acid sequence comprising SEQ ID NOs 4 and 6 joined by a synthetic amino acid linker.

28) (canceled)

29) (canceled)

30) (canceled)

31) The polypeptide of claim 27, wherein said polypeptide binds to a mammalian cell surface receptor.

32) The polypeptide of claim 31, wherein the polypeptide comprises SEQ ID NOs 2 and 4 joined by a synthetic amino acid linker, and wherein the mammalian cell surface receptor is IL-23 receptor.

33) The polypeptide of claim 32, wherein the Il-23 receptor is primate IL-23 receptor.

34) The polypeptide of claim 33, wherein the primate is cynomolgus macaque.

35) The polypeptide of claim 34, wherein the polypeptide binds to both the human and cynomolgus macaque IL-23 cell surface receptor.

36) A composition comprising the polypeptide of claim 27 and a sterile buffer solution.

37) (canceled)

38) (canceled)

39) The polypeptide of claim 31, wherein the polypeptide comprises SEQ ID NOs 4 and 6 joined by a synthetic amino acid linker, and wherein the mammalian cell surface receptor is IL-12 receptor.

40) The polypeptide of claim 39, wherein the mammalian IL-12 receptor is a primate IL-12 receptor.

41) The polypeptide of claim 40, wherein the primate is cynomolgus macaque.

42) The polypeptide of claim 41, wherein the polypeptide binds to both the human and cynomolgus macaque IL-12 cell surface receptor.

43) (canceled)

44) The polypeptide of claim 27, immobilized on a solid support.

45) The polypeptide of claim 44, wherein the solid support is selected from the group consisting of: a nitrocellulose filter, a bead, a multiwell plate, and a chip.

46) (canceled)

47) (canceled)

48) A binding compound which binds to a polypeptide comprising a sequence selected from an amino acid sequence comprising SEQ ID NOs 2 and 4 joined by a synthetic amino acid linker, and an amino acid sequence comprising SEQ ID NOs 4 and 6 joined by a synthetic amino acid linker and modulates its activity.

49) The binding compound of claim 48, wherein the binding compound inhibits the activity of the polypeptide.

50) The binding compound of claim 48, wherein the binding compound stimulates the activity of the polypeptide.

51) The binding compound of claim 48, wherein the binding compound is selected from the group consisting of an antibody, an aptamer, or a small molecule.

52) (canceled)

53) (canceled)

54) (canceled)

55) (canceled)

56) A method of identifying a binding compound, comprising the steps of:
a) contacting a binding compound with a cynomolgus macaque polypeptide comprising a sequence selected from the amino acid sequence of SEQ ID NO 2, the amino acid sequence of SEQ ID NO 4, the amino acid sequence of SEQ ID NO 6, an amino acid sequence comprising SEQ ID NOs 2 and 4 joined by a synthetic amino acid linker, and an amino acid sequence comprising SEQ ID NOs 4 and 6 joined by a synthetic amino acid linker;
b) selecting the binding compound that binds to the cynomolgus macaque polypeptide to result in a candidate binding compound,
c) contacting the candidate binding compound with a human polypeptide selected from the group consisting of: p19, p40, p35, IL-23 or IL-12; and
d) identifying the candidate binding compound that binds to both the cynomolgus macaque polypeptide and its human homolog.

57) A method of identifying a binding compound, comprising the steps of:
a) contacting a binding compound with a cynomolgus macaque polypeptide comprising a sequence selected from the amino acid sequence of SEQ ID NO 2, the amino acid sequence of SEQ ID NO 4, the amino acid sequence of SEQ ID NO 6, an amino acid sequence comprising SEQ ID NOs 2 and 4 joined by a synthetic amino acid linker, and an amino acid sequence comprising SEQ ID NOs 4 and 6 joined by a synthetic amino acid linker;
b) selecting the binding compound that modulates a function of the cynomolgus macaque polypeptide to result in a candidate binding compound,
c) contacting the candidate binding compound with a human polypeptide selected from the group consisting of: P19, P40, P35, IL-23 or IL-12; and
d) identifying the candidate binding compound that modulates the function of both the cynomolgus macaque polypeptide and its human homolog.
